(19) United States
(12) Patent Application Publication
(10) Pub. No.: US 2023/0145185 A1
(43) Pub. Date: May 11, 2023

(54) CELL SORTING MICROBEADS AS NOVEL CONTRAST AGENT FOR MAGNETIC RESONANCE IMAGING

(71) Applicant: University of Kentucky Research Foundation, Lexington, KY (US)

(72) Inventors: Fanny Chapelin, Lexington, KY (US); Roberto Gedaly, Lexington, KY (US); Aman Khurana, Lexington, KY (US); Francesc Marti, Lexington, KY (US)

(21) Appl. No.: 18/054,387

(22) Filed: Nov. 10, 2022

Related U.S. Application Data
(60) Provisional application No. 63/277,843, filed on Nov. 10, 2021.

(51) Int. Cl. A61K 49/16; C12N 5/0783; C12N 5/00; A61K 49/18

(52) U.S. Cl. CPC A61K 49/16; C12N 5/0636; C12N 5/0006; A61K 49/1875

(57) ABSTRACT

The present disclosure generally relates to methods and compositions for obtaining magnetic resonance images of labelled cells. The methods include internalizing a superparamagentic iron oxide nanoparticle within a desired population of cells and then observing the cells through the contrast provided in magnetic resonance imaging. The methods are applicable for in vivo use to monitor desired cells types.

### CROSS-REFERENCE TO RELATED APPLICATIONS

This application for patent claims priority to U.S. Provisional Patent Application 63/277,843, filed Nov. 10, 2021, the contents of which are hereby incorporated by reference in their entirety.

### GOVERNMENT SUPPORT

The present disclosure was supported by grants from the National Institutes for Health (Grant Nos. P20CA177558 and UL1TR001998). The Government may have rights to the invention.

### TECHNICAL FIELD

The present disclosure generally relates to providing superparamagnetic iron oxide to cells to provide a mechanism for the tracking and observation thereof.

### BACKGROUND

Rapid evolution and prevalent use of magnetic resonance imaging (MRI) in the clinic has fueled research in the development of contrast agents and methods to improve pathological tissue delineation and to visualize specific cell trafficking in vivo. The first reported use of contrast agents in human MRI studies employed oral ferric chloride to monitor bowel metabolic processes. Nowadays, 25-40% of all clinical MRIs employ contrast agents for tissue enhancement and numerous clinical studies have proven the feasibility of longitudinal tracking of specific cell populations.

MRI signal intensity and contrast is controlled by numerous intrinsic factors, including spin density, tissue susceptibility, T1 (longitudinal) and T2 (transverse) relaxation times and physiological motion. Extrinsic contrast agents usually target proton relaxation. They are composed of paramagnetic gadolinium, manganese ion complexes or superparamagnetic iron oxide particles nanoparticles (SPIO). Although gadolinium chelates are currently the only clinically available option, increased toxicity reports and lack of labeling specificity have rekindled interest for new and more effective agents. Accumulation of superparamagnetic iron oxides (SPIOs) in tissue provides excellent contrast by altering the relaxation times of adjacent protons after applying RF pulses. Subsequently produced T1/T2 shortening effects results in intense negative contrast which aids in differentiating these tissues from the adjacent normal tissue signals.

Ferumoxytol (Feraheme) is an FDA approved iron-rich nanoparticle originally designed for intravenous treatment of patients with iron deficiency. Following uncovering of its MR properties, ferumoxytol has been extensively used off-label for cell tracking in numerous applications, such as cancers, immune disorders, and cell transplant imaging in preclinical studies. Cell types presenting with low phagocytic activity, such as T cells, are not easily labeled with existing contrast agents like ferumoxytol. Additionally, in vitro manipulations to increase cell labeling also exacerbates risks of cell death or transformation. Reducing in vitro handling also directly affects time and sterility concerns when using Tregs (Regulatory T cells) in a transplant surgery setting. Accordingly, there is a need for improved approaches for labeling and monitoring cells.

### SUMMARY

The present disclosure generally relates to providing superparamagnetic iron oxide to cells to provide a mechanism for the tracking and observation thereof.

In some aspects, the present disclosure concerns a method for obtaining a magnetic resonance image (MRI) of a cell through obtaining a cellular sample and binding to one or more cells therein a nanoparticle that includes an antibody or active fragment thereof to a cell surface protein. In some aspects, the antibody or active fragment thereof is covalently linked to at least one superparamagnetic iron oxide nanoparticle. In some aspects, the one or more cells are allowed to incubate with the nanoparticles for a period of time from about 5 minutes to 24 hours or more. In some aspects, the method may include stimulating the one or more cells with an agonist to cause the nanoparticle to be internalized. In some aspects, the methods include then obtaining at least one MRI of the one or more cells.

In some aspects, the methods of the present disclosure may include the cellular sample being obtained from a subject, such as a human or mammalian subject.

In some aspects, the methods may further include administering the one or more cells back to the subject prior to obtaining the at least one MRI or to another subject prior to obtaining the at least one MRI.

In some aspects, the methods may include administering the one or more cells to a subject prior to obtaining the at least one MRI.

In some aspects, the methods of the present disclosure may include one or more steps for isolating the one or more cells from the cellular sample.

In some aspects, the antibody or active fragment thereof binds to CD25. In certain aspects, the antibody or active fragment thereof binds to human CD25. In further aspects, the agonist is CD3, CD28, IL-2, or a combination thereof.

In some aspects, the antibody or active fragment thereof binds to CD4. In certain aspects, the antibody or active fragment thereof binds to human CD4. In further aspects, the agonist is CD3, CD28, IL-2, or a combination thereof.

In some aspects, incubating may include providing a cellular medium and about 37° C. temperature to the one or more cells.

In some aspects of the methods of the present disclosure, the one or more cells are T cells. In further aspects, the T cells are Treg (T regulatory) cells.

In some aspects, the agonist may be covalently linked to a solid support, such as a microparticle.

In some aspects, the present disclosure concerns a nanoparticle for MRI that includes of a superparamagnetic iron oxide nanoparticle covalently linked to an antibody or active fragment thereof, wherein the antibody or active fragment thereof recognizes a cell surface protein. In some aspects, the antibody or active fragment thereof binds to CD25 or CD4.

### BRIEF DESCRIPTION OF THE DRAWINGS

FIG. 1A depicts magnetic resonance signal effects of CD3 and CD25 microbeads compared to ferumoxytol. T2-weighted magnetic resonance images (upper row) and corresponding T2* relaxation time (lower row) maps of agarose gels containing increasing bead concentrations (right to left) show marked signal decrease (dark) for all contrast agents compared to controls (*=agarose gel alone). Signal decay is more pronounced for CD25 microbeads and ferumoxytol compared to CD3 microbeads. Data was acquired on a 1.5 T MRI.

FIG. 1B depicts magnetic resonance signal effects of CD25 microbeads compared to ferumoxytol. T2-weighted magnetic resonance images of agarose gels (200 μl) containing increasing contrast agent concentrations (right to left: 0, 12.5, 25, 50 or 100 μg/ml of CD25 microbeads or ferumoxytol) show marked signal decrease (dark) for both contrast agents compared to controls (0=agarose gel alone). Signal decay is more pronounced for CD25 microbeads compared to ferumoxytol. Data was acquired on a 7 T MRI with 10 TEs equally spaced from 6.4 to 64 ms

FIG. 2 depicts Quantitative T2 decay as a function of iron concentration and relaxivity measurement. (a) shows T2 values for CD25 microbeads and ferumoxytol follow a decreasing curve with increasing iron amount. T2 decay is more pronounced in CD25 microbead samples compared to ferumoxytol samples. Data are presented as ROI mean±standard deviation. (b) shows the inverse of T1 values were plotted against iron concentration to yield CD25 microbead and ferumoxytol r₁. (c) shows the inverse of T2 values were plotted against iron concentration to yield CD25 microbead and ferumoxytol r₂. CD25 microbeads display much larger r₂/r₁ ratio than ferumoxytol (r₂/r₁=468 and 52 respectively).

FIG. 3 depicts T2* relaxation curves of microbeads. CD25 microbeads and ferumoxytol display short T2* relaxation, enabling better contrast from surrounding water. In contrast, CD3 microbeads and agarose gel exhibit more linear signal decay with respect to echo time.

FIG. 4 depicts confirmation of CD25 microbead intracellular uptake by Tregs. Regulatory T cells labeled overnight with 10 μl CD25 microbeads (45 μg Fe/ml) exhibit positive intracellular staining (a, arrows) (staining with Prussian blue). Tregs labeled with ferumoxytol overnight (b) do not internalize the contrast agent. Unlabeled control Treg (c) and T cells (d) do not exhibit staining.

FIG. 5 depicts magnetic resonance signal effects of CD25 microbead-labeled Tregs. T2-weighted magnetic resonance images (FIG. 5A) and corresponding T2 relaxation time maps (FIG. 5B) of agarose gels containing CD25 microbead-labeled Tregs (1-2) show marked signal reduction compared to unlabeled T cells (3-4) and agarose (5) controls. Quantitative analysis of the T2 relaxation times (FIG. 5C) confirms significant T2 shortening in CD25-microbead labeled cells. T2 of half million Tregs is about three-fourth as high as the T2 of unlabeled T cells and agarose alone. T2 is further shortened in one million labeled Treg samples (1). Data are presented as mean±standard deviation. * indicates p<0.001 compared to agarose control.

FIG. 6 depicts histological assessment of nanoparticle uptake by macrophages. Light microscopy images of macrophages labeled with varying concentrations of CD25 microbeads (top row) and ferumoxytol (bottom row) are displayed. Data shows strong intracellular localization of ferumoxytol, conversely to CD25 microbeads, suggesting that the CD25 antibody does not support internalization of the beads by unspecific phagocytic cells/macrophages. Scale bar represents 100 μm.

FIG. 7 depicts axial T2-weighted liver MR images of mice pre and 24 h post-intravenous injection of labeled cells or CD25 microbeads. Representative images of mouse receiving CD25 microbead-labeled Treg cells pre (a) and 24 h-post (b) IV injection show T2 signal reduction in the liver (arrow) compared to mouse receiving CD25 microbead-labeled T cells (c and d, negative control). Positive control mice receiving CD25 microbeads alone intravenously show marked T2 signal decrease in the liver (arrow) 24 h post-injection (e,f).

FIG. 8 depicts quantitative summary of in vivo T2 measurements. T2 relaxation times show significantly shorter T2 values in Treg-receiving mice 24 h post-injection compared to prescan (p=0.007). On the contrary, T cell-receiving mice show no differences in T2 (p=0.44). Positive control mice receiving CD25 microbeads alone show drastic T2 reduction as expected. The difference between Treg and T cell receiving mice is also significant (p=0.029). Data are displayed as T2 means for each animal (9 measurements per animal per data point).

FIG. 9 depicts the sequential gating strategy used to identify resting (CD25⁻) conventional primary human CD4⁺ T cells (Tconv), activated (CD25⁺/FoxP3^{-/LOW}) Tconv and Treg cells from peripheral blood mononuclear cells is based on the differential expression of CD25 and FoxP3 markers in CD4⁺ T cells. The identification of Treg cells required a further analysis of CD127 expression and were identified as CD4⁺/CD25⁺/FoxP3⁺/CD127^{-/LOW}

FIG. 10 depicts cargo-induced endocytosis of IL2-R in CD25 nanoparticle-labeled Treg cells. Drug treatments do not affect Treg cell viability. Freshly isolated, non-activated, CD4⁺ T cells were segregated into membrane-expressing CD25⁻ conventional (Tconv), CD25⁺ Tconv and (FoxP3⁺/mCD25⁺/CD127⁻) Treg cells as in FIG. 1. Cells were incubated with or without 1 μL of CD25 reagent (Miltenyi)/10⁶ cells for 10 minutes with slow shaking at 4° C. (same conditions used for Treg cell isolation). After washing off the excess reagent, cells were left in culture for additional 6 hours, harvested and analyzed by flow cytometry. The results showed that Treg cells that were in the presence of CD25 reagent, expressed twice as much internalized CD25 marker (IL-2 Receptor α subunit) compared to cells without reagent. The same effect was observed in CD25⁺ Tconv cells. The table included illustrates that the data of the different cell types were acquired from the same tube.

FIG. 11 depicts quantitative analysis of the T2 shortening in CD4-microbead labeled cells. Data are presented as mean±standard deviation. * indicates p<0.001 compared to agarose control.

FIG. 12 depicts confirmation of CD4 microbead intracellular uptake by Tregs. Unlabeled control Treg (bottom image) do not exhibit staining.

FIG. 13 depicts electron microscopy data confirming intracellular localization of CD4 microbeads. In the top left is presented a full cell image. The bottom left shows the magnified inset thereof. The bottom right shows an iron-specific filter. The top right shows an inset EDS spectrum showing presence of iron in the cell.

### DESCRIPTION

The present disclosure concerns methods and compositions for cell labelling to provide a means for tracking or locating cells. In some aspects, the methods and compositions herein allow for providing a label and/or tracing material to an internal portion of a cell, as well as steps to monitor or observe the presence of the label and/or tracking material for a prolonged period of time. In some aspects, the label and/or tracking material can be monitored and/or observed through magnetic resonance imaging (MRI). In some aspects, the label and/or tracking material is a superparamagnetic material. In some aspects, the label and/or tracking material is a nanoparticle or microparticle of from about 5 nm in diameter to about 2 μm (3500 nm) in diameter, including about 10, 20, 30, 40, 50 60, 70, 80, 90, 100, 200, 300, 400, 500, 600, 700, 800, 900, 1000. 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, and 3400 nm in diameter. In some aspects, the label and/or tracking material is a nanoparticle or microparticle composed of or including superparamagnetic iron oxide. In some aspects, the nanoparticle or microparticle may further include a coating, such as a saccharide or polysaccharide coating. In some aspects, the nanoparticle or microparticle may be coated with dextran.

In some aspects, the present disclosure concerns nanoparticles or microparticles affixed with one or more antibodies or active fragments thereof. As will be appreciated, an active fragment of an antibody includes peptide fragments of a heavy and or light chain of an antibody that retain the ability to bind and/or recognize the epitope of the full antibody. Such antibodies and active fragments may include immunoglobulin (Ig) G, IgA, IgM, IgE, Fab, F(ab′)2, monospecific Fab2, bispecific Fab2, trispecific Fab2, movovalent Igs, scFv, diabodies, triabodies, scFv-Fc, minibodies, heavy-chain only Igs, V_{H} domains, V_{L} domains, and V_{HH}.

In some aspects, the antibodies or active fragments thereof are directed to binding a particular epitope. In some aspects, the epitope is an extracellular epitope, such as a cell surface receptor. In some aspects, the antibody or active fragment thereof may be specific to a cell receptor states, such as an active and/or dimerized and/or trimerized receptor. In some aspects, the antibody or active fragment thereof binds to a receptor epitope. In some aspects, the antibody or active fragment thereof binds to a subunit of a receptor. In some aspects, binding of the antibody or active fragment thereof allows for the nanoparticle or microparticle to be effectively bound to a cell surface protein, such as a cell surface receptor or a subunit thereof.

In some aspects, the present disclosure concerns binding a solid nanoparticle or microparticle to a cell surface protein, such as a receptor, through the intermediary antibody or active fragment thereof. Through the use of antibodies or active fragments thereof, the binding of the solid nanoparticle or microparticle to the cell is specific for the expression of the epitope recognized by the antibody or active fragment thereof. Accordingly, the nanoparticle or microparticle can be affixed to a particular cell or cell type based on the presence of the epitope being exclusive to a particular cell or cell type.

In some aspects, the nanoparticles or microparticles are accordingly incubated with a population of cells to allows for the nanoparticle or microparticle to bind to one or more particular cells or cell types within the population based on the presence of the recognized epitope on the exterior of certain cells within the population. In some aspects, the epitope is on a cell surface protein or receptor. In some aspects, the cell surface protein or receptor is capable of being internalized by endocytosis. In some aspects, the cell population is in vitro. In some aspects, the cell population is in vivo. In some aspects, the cell population is ex situ from a subject, such as a sample or excised tissue.

In some aspects, the antibody or active fragment thereof binds to a cell marker of a particular cell type or types. For example, stem cells can be labelled through CD34, and CD117, leukocytes can be labelled through CD45, granulocytes can be labelled by binding CD45, CD11b, CD15, CD24, CD114, and CD182, monocytes through CD4, CD45, CD14, CD114, CD11a, CD11b, CD91, and CD16, T lymphocytes by CD45 and CD3, T helpers through CD45, CD3, and CD4, Tregs by CD4, CD25, and FOXP3, cytotoxic T cells by CD45, CD3, and CD8, B lymphocytes through CD45, CD19, CD20, CD24, CD38, and CD22, thrombocytes by CD45 and CD61, and NK cells by CD16, CD56, CD31, CD30, and CD38.

In some aspects, the present disclosure concerns labeling or affixing the nanoparticle or microparticle to a subset of cells within a population. It will be appreciated that the selection of the antibody or active fragment thereof can determine which cells or cell types are labelled or affixed with the nanopartricle or microparticle. In some aspects, the label and/or tracking material of the nanoparticle or microparticle can be utilized to separate the labelled cells. In some aspects, the label and/or tracking material can be utilized to visualize or observe cells bound to the nanoparticle or microparticle. In some aspects, the label and/or tracking material is internalized within the cell, thereby allowing the cell to be identified by the internal presence of the label and/or tracking material.

In some aspects, the utilization of superparamagnetic iron oxide as the label or tracking material can provide for both isolating and observing cells expressing the epitope to the antibody or active fragment thereof of the nanoparticle or microparticle.

In some aspects, the nanoparticles can be utilized to isolate a cell, such as through chromatograph means. The step of utilizing an antibody or active fragment thereof tether to a solid support is generally understood. By passing or incubating a population of cells or cellular material with an antibody or active fragment thereof affixed to a solid support, where present, the epitope is bound and the cell or protein expressing the epitope is immobilized to the solid support. Such allows for washing or removing cells or proteins that lack the epitope. In some aspects, the epitope can be released from antibody binding by means such as with competitive binding or high salt concentration elution.

In some aspects, the inclusion of superparamagnetic iron oxide as the labelling or tracking material of the nanoparticle or microparticle allows for application of a magnetic field to isolate cells or proteins expressing the epitope bound to the antibody or active fragment thereof. The use of magnetic fields for such process is generally understood in the art, such as with magnetic-activated cell sorting (MACS). MACS is a method for cell population separation from tissue homogenates. The technique relies on targeting cell surface markers with specific antibodies bound to superparamagnetic iron oxide of the nanoparticles or microparticles (sometimes referred to in the art as ‘microbeads’). When placed in a high gradient magnetic field, the labeled cells remain trapped while unlabeled cells elute. Labeled cells can then be collected by removing the magnetic field.

In some aspects, the present disclosure concerns providing conditions and/or stimuli for the nanoparticle or microparticle to be internalized within the cell to which it is attached, such as by stimulating endocytosis. Following binding of the nanoparticle or microparticle to the cell, the internalization thereof allows for the labelling or tracking material to be internalized within the cell. It will be appreciated that in some aspects, the step of isolating cells may not be required as the binding of the nanoparticle or microparticle is specific to the presence of the antibody or active fragment thereof.

In some aspects, the nanoparticle or microparticle can be internalized by incubating the cell population or isolated cell population for a period of time from about 5 minutes to about 24 hours or longer, including about 10 mins, 15 mins, 20 mins, 25 mins, 30 mins, 40 mins, 50 mins, 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 124 hrs, 15 hrs, 16 hrs, 17 hrs, 18 hrs, 19 hrs, 20 hrs, 21 hrs, 22 hrs, and 23 hrs or more. In some aspects, the antibody or active fragment thereof can bind sufficiently to a cell surface protein such that the cell surface protein signals for the plasma membrane to engulf the cell surface protein and internalize the cell surface protein. In some aspects of the present disclosure, as set forth herein, it is identified that the process of endocytosis of a cell surface protein will endocytose the bound nanoparticle or microparticle.

In some aspects, the cell surface protein may recognize the bound antibody or active fragment thereof as an agonist and internalize the cell surface protein in response. In further aspects, the addition of an agonist or stimulus for the cell surface protein may trigger endocytosis of the cell surface protein after the nanoparticle or microparticle is bound.

By way of example, the Examples section herein details the isolation and internalization of nanoparticles or microparticles of superparamagnetic iron oxide in Regulatory T cells (Tregs). A population of blood cells is obtained and then incubated with superparamagnetic beads affixed with a CD25 antibody and/or CD4 antibody. The CD25 antibody recognizes CD25 expressed on cell surfaces, which is one of the markers upregulated in Tregs and, to a lesser extent, to activated T cells, thereby allowing, under appropriate conditions, for the selective binding of the nanoparticle or microparticle to Treg cells. Similarly, the CD4 antibody recognizes CD4 expressed on cell surfaces, which is a further marker upregulated in Tregs. Once the nanoparticle or microparticle is bound to the Treg cell, the methods of the present disclosure allow for internalization of the nanoparticle or microparticle within the Treg cell. Such may be accomplished through a deliberate incubation period, providing a stimulus to the Treg cells, or a combination of both incubation and stimulation.

In some aspects, the internalization of the nanoparticle or microparticle can be achieved by incubating the cell for a determined period of time. It will be appreciated that incubation involves a temperature at or near body temperature. For example, human Treg cells bound to the nanoparticles or microparticles can be incubated at about 37° C. Incubation may further be in a liquid medium, such as a cell culture medium.

In some aspects, incubation alone is sufficient to internalize the nanoparticle or microparticle. In some aspects, internalization may be achieved or enhanced by including a stimulus or activator to the cell. In some aspects, the Treg cells can be stimulated or activated, such as by adding a known cytokine or growth factor. For example, for Treg cells, such may include interleukin-2 (IL-2), CD3, CD28, or combinations thereof. In some aspects, the stimulus or agonist for activation may itself be attached to a solid support such as another nanoparticle or microparticle. Such a solid support may provide a mechanism for removing the stimulus or agonist, such as by centrifugation or gravitational pull. The length of time for stimulation and dose required may vary depending on the concentration of cells, as well as the cell surface protein/receptor. In some aspects, the cells can be stimulated or activated while being incubated, such as introducing an agonist to Treg cells while the cells are maintained at 37° C.

The internalization of the label and/or tracking material within a particular cell then allows for the tracking or observation of the cell or of a population of cells with internalized labels and/or tracking materials. For example, as with above, internalization of superparamagnetic iron oxide within a Treg cell allows for the Treg cell to be identified or observed through detecting the internalized label. For example, superparamagnetic iron oxide exhibit unique magnetic properties that allows them to serve as a contrast agent during magnetic resonance imaging (MRI). In some aspects, the size and surface properties of the iron oxide particles may determine whether the internalized nanoparticle or microparticle provides a contrasting view at T1 or T2 weighted images during an MRI acquisition.

In some aspects, the present disclosure concerns applying a magnetic field to cells with the nanoparticles or microparticles internalized therein. It will be appreciated that in some aspects, application of a magnetic field can be utilized to attract the cell. In further aspects, the internalization of superparamagnetic iron oxide particles within a cell provides a contrast agent within the cell during magnetic resonance imaging.

In some aspects, the present disclosure concerns obtaining an MRI image of the cell with the nanoparticle or microparticle internalized therein. In some aspects, the MRI image is obtained in vitro. In further aspects, the cells with the internalized nanoparticle or microparticles can be administered to a subject and an MRI image is obtained at one or more time points thereafter. In some aspects, the cells are administered to the subject from which the cells were obtained, derived or isolated. In some aspects, a subject may receive non-autologous labelled cells. It will be appreciated that in such aspects, the cells can be screened or analyzed to avoid rejection. In some aspects, the subject receives allogenic labelled cells.

In some aspects, the labelled cells are T cells. In some aspects, the labelled cells are Treg or CD25+ cells or CD4+ cells. In some aspects, the present disclosure concerns obtaining a sample from a subject, labelling Treg cells or other CD25+ or CD4+ cell subset within the sample with a nanoparticle or microparticle as set forth herein, causing the nanoparticle or microparticle to be internalized in the Treg cells or CD25+ cells or CD4+ cells and administering the Treg cells or CD25+ cells or CD4+ cells back to the subject. In some aspects, the Treg or CD25+ cells or CD4+ cells are incubated at about 37° C. to internalize the nanoparticle or microparticle and then administered back to the subject or to a different subject. In some aspects, the Treg or CD25+ cells or CD4+ cells are activated prior to administration back to the subject or to another subject. In some aspects, the Treg or CD25+ cells or CD4+ cells are incubated and activated prior to administration back to the subject or to another subject. In some aspects, the methods further include obtaining one or more MRI images from the subject to determine the location and/or concentration of the labelled Treg or CD25+ cells or CD4+ cells. It will be appreciated that obtaining the localization and/or concentration of the labelled Treg cells allows those skilled in the art to correlate changes in location and/or concentration to overall Treg cell activity within the subject. Such can be of clinical significance in determining the overall homeostasis and self-tolerance of the subject, as well as identifying issues with regard to autoimmunity, tumor immunity, and transplant acceptance/rejection.

### EXAMPLES

In this study, a comparison between the MR signal effects of MACS microbeads (CD25) and in vitro activation beads (CD3/28) to the preclinical off label gold standard ferumoxytol is performed.

Materials and Methods

Contrast Agents

Microbeads (CliniMACS CD25, Miltenyi Biotec, Somerville, Mass.) for magnetic-assisted cell sorting (MACS) are composed of a CD25 antibody tethered to a dextran-coated iron bead. Here, CD25 microbeads were used for positive regulatory T cell (Treg) sorting. The beads average 50 nm in diameter.

CD2/3/28 microbeads (T cell activation/expansion kit, Miltenyi Biotec) are physiological activation beads generally used for T cell activation and display CD3 or CD28 antibodies bound to a 3.5 μm iron bead.

Ferumoxytol (AMAG Pharmaceuticals, Waltham, Mass.) nanoparticles are caboxydextran-coated iron cores with a mean diameter of 30 nm. Ferumoxytol's magnetic properties and its use for cell labeling and subsequent tracking is well studied. Ferumoxytol's relaxivities in saline are r₁=19.9 s⁻¹ mM⁻¹ and r₂=60.8 s⁻¹ mM⁻¹ respectively at 1.5 T.

Bead Phantom Preparation

Phantoms were prepared by diluting different amounts of CD25 microbeads or ferumoxytol in 1% agarose for a final volume of 200 μl. Concentrations tested included 0, 12.5, 25, 50 and 100 μgFe/ml. Dilutions were chosen based on in vitro working volumes that would be suitable for preserved cell viability. For relaxivities measurements, phantoms were prepared by diluting different amounts of CD25 microbeads or ferumoxytol in PBS for a final volume of 200 μl. Concentrations tested included 0, 3.1, 6.25, 12.5, 25 and 50 μgFe/ml. Dilutions were chosen to obtain optimal recovery curves for T1 and T2 measurement at different concentrations.

MRI Measurements of Bead Phantoms on Clinical Scanner

All MRI images in this study were acquired on a preclinical Bruker BioSpec 7 T imaging system (Bruker, Billerica, Mass.) using a mouse birdcage body coil (Bruker). Axial images of phantoms were acquired using a Turbo RARE sequence with parameters TR/TE=2000/20 ms, Matrix size=96×96, FOV=36×36 mm², Slice thickness=1.2 mm, Averages=2, Slice number=16. A T2map Multi Slice Multi Echo (MSME) sequence with TR=2000 ms and 10 TEs equally spaced from 6.4 to 64 ms, Matrix size=96×96, FOV=36×36 mm², Slice thickness=1.2 mm, Averages=3, Slice number=16 was acquired for T2 and r₂ calculations. A T1map RARE sequence with TE=25.7 ms and 8 TRs equally spaced from 900 to 5000 ms, Matrix size=256×192, FOV=36×26 mm², Slice thickness=1.2 mm, Averages=1, Slice number=16 was acquired for r₁ calculations. T1 and T2 values were extracted using the system's inbuilt Image Sequence Analysis (ISA T2vtr and T1sat, Paravision 360, V3.0) software by defining three circular ROIs in the center of the PCR tubes.

Regulatory T Cell Isolation

Peripheral blood mononuclear cells were isolated from buffy coats of healthy anonymous blood donors (Kentucky Blood Center, Lexington, Ky.) in accordance with relevant guidelines and regulations via Ficoll density gradient (Histopaque 1077, Sigma Aldrich, St Louis, Mo.). Informed consent was obtained from all participants. T cells were isolated via EasySep Human T cell Isolation Kit (Stemcell Technologies, Cam-bridge, Mass.), and CD25 Microbeads II (Miltenyi Biotec) were used to further isolate CD25⁺ T cells (here referred to as Tregs to model future Treg isolation). Treg and T cells (CD25 negative) were maintained in RPMI media (Corning, Corning, N.Y.) supplemented with 10% fetal bovine serum (Hyclone, GE Healthcare, Marlborough, Mass.), 1% penicillin streptomycin (VWR, Radnor, Pa.), and 100 units/ml of recombinant human interleukin 2 (IL-2, Peprotech, Rocky Hill, N.J.). On the first day of culture, cells were activated with T cell Transact (Miltenyi Biotec) per manufacturer's instructions. Cells were expanded for up to 14 days to obtain cell amounts needed.

Regulatory T Cell Labeling and Histological Staining

To optimize labeling efficacy, Tregs and T cells (CD25−) were plated at a density of 2 million cells per ml of full media in 24 well plates in triplicates. CD25 microbeads were added to the culture media in varying amounts (0, 10, 20 μl per million cells) and several incubation times (0, 6, 16 hours) were tested. Triplicates of Tregs labeled with Ferumoxytol with final concentration of 400 μgFe/ml were also prepared. Triplicates of unlabeled Tregs and T cells were also prepared as controls. After incubation, excess label was removed by three washes followed by centrifugation. T cells were resuspended at a density of 1 million cells in 100 μl phosphate buffered saline and gently spread on a histology slide (Superfrost slide, Fisherbrand, Waltham, Mass.). Samples were allowed to air dry and were subsequently fixed with 10% formalin (VWR, Radnor, Pa.) for 15 minutes. Samples were then stained using a Prussian blue iron staining kit (Polysciences, Warrington, Pa.) consisting of 15 minute incubation of 1:1 potassium ferrocyanide and hydrochloric acid followed by 5 minutes of nuclear fast red counterstain. Samples were thoroughly washed and allowed to fully air dry. Coverslips were secured onto the slide with Permount (Fisher Scientific, Waltham, Pa.) mounting media. Histological images were acquired on a Nikon Ti-U Microscope (Nikon, Tokyo, Japan) with 20× and 60× objectives.

Macrophage Labeling and Histological Staining

TIB-67 murine macrophage cells were kindly provided by Melissa Hollifield and cultured in DMEM media supplemented with 10% fetal bovine serum and 1% penicillin streptomycin. Triplicate samples of 40,000 cells were plated in chamber slides at a density of 40,000 cells/cm². ferumoxytol was added to each well to reach a final concentration of 200 (low) or 400 (high) μgFe/ml and one well did not receive ferumoxytol and served as control. In other arrangements, 10 (low) or 20 (high) μl of CD25 microbeads were added to each well and one beadless well served as control. Chamber slides were kept in the incubator at 37 degrees and 5% CO₂ overnight. Excess contrast agent was aspirated with the media and chamber slides were washed three times before proceeding to histological staining. Samples were fixed and stained for iron and imaged as described above.

MRI Measurement of Regulatory T Cells Labeled with CD25 Beads

Triplicate samples of half and one million Treg were labeled overnight (16 hours) with 10 μl CD25 microbeads per million Tregs. After incubation, excess microbeads were removed by three washes followed by centrifugation. Triplicate samples of unlabeled half and one million T cells were also prepared. All cell samples were resuspended in 100 μl agarose gel (1% dilution in PBS) and transferred to PCR tubes for MR imaging.

In Vivo MRI Tracking of CD25 Microbeads-Labeled Tregs

Fresh human Tregs (CD25+) cells were incubated for 6 h with 10 μl CD25 microbeads per million Tregs (45 μgFe/ml) and maintained at a concentration of 2×10⁶ cells/ml of full media in 6-well plates. After incubation, excess microbeads were removed by three washes followed by centrifugation. Fresh human T cells (CD25−) were prepared under the same conditions. Cell samples were resuspended at a concentration of 1.5×10⁷ cells in 100 μl PBS for intravenous injection. Twelve six to eight week-old female NSG mice were sourced from Jackson Labora-tory (strain #005557). Liver MRI prescans were acquired for all mice prior to intravenous injections. Five mice received CD25 microbead-labeled Tregs, five mice received CD25 microbead labeled T cells (negative control) and 2 mice received CD25 microbeads alone (20 μl diluted in 80 μl PBS, final concentration of 450 μgFe/ml, positive control). Liver MRIs were acquired again 24 h post-injection.

MRI images were acquired on a preclinical Bruker 7T imaging system (Bruker, Billerica, Mass.) using a mouse birdcage body coil (Bruker). Axial images of the cell samples were acquired using a Turbo RARE sequence with parameters TR/TE=2500/33 ms, Matrix size=128×128, FOV=28×28 mm², Slice thickness=0.7 mm, Averages=2, Slice number=16 and a T2map Multi Slice Multi Echo (MSME) sequence with TR=2200 ms and TEs ranging from 7 to 90 ms, Matrix size=96×96, FOV=28×28 mm², Slice thickness=0.7 mm, Averages=1, Slice number=16. T2 values were extracted using the system's inbuilt Image Sequence Analysis (ISA T2vtr) software by defining circular ROIs over each tube and averaged over 3 slices.

Statistical Analyses

Statistical comparisons between phantoms and cell samples were performed using a t-test. P values less than 0.05 were considered significant. For in vivo experiments, a total of 216 T2 observa-tions from 12 mice were available for analysis. Statistical significance was set at p≤0.05p≤0.05 and all tests were two-sided. Missing observations were reported and were excluded on an analysis-by-analysis basis. Paired t-tests were performed for comparisons of prescan and 24-h T2 measurements within treatment group and a one-way ANOVA was performed for comparisons between groups. All analyses were done in R programming language, version 4.1.2 (R Foundation for Statistical Computing, Vienna, Austria). All graphics were produced using the R package ggplot2, version 3.3.5 (Hadley Wickham).

Results

Microbeads MRI Characteristics

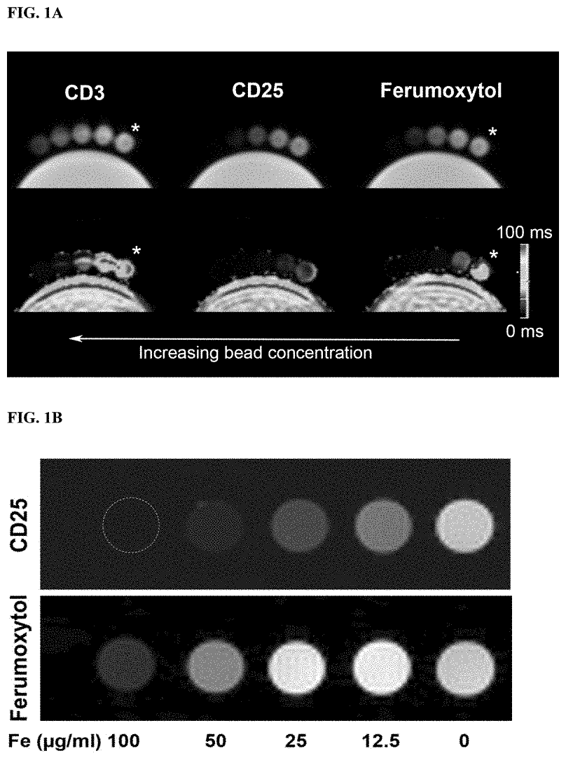

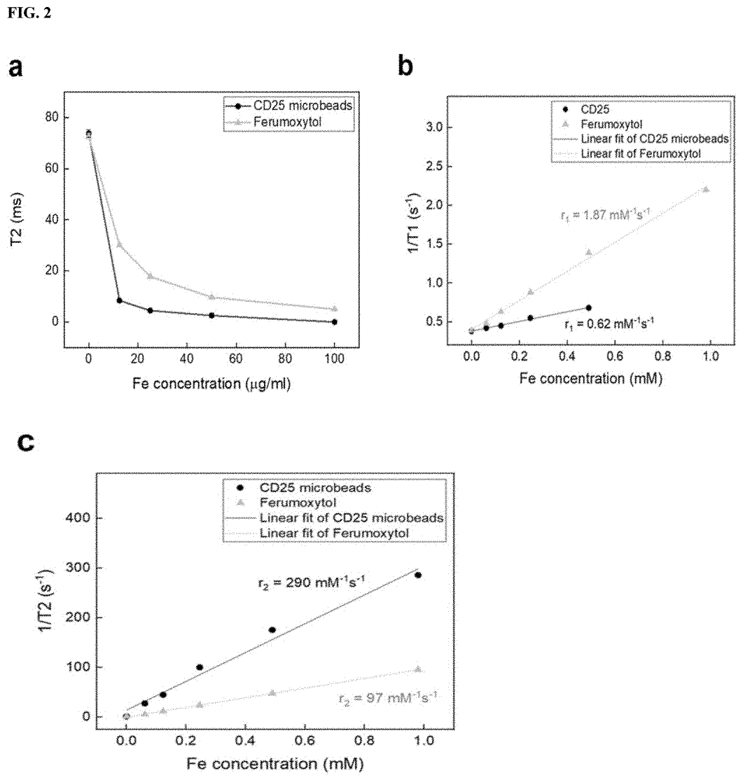

The respective magnetic properties of CD3 and CD25 microbeads were tested compared to cell tracking standard Ferumoxytol (FIG. 1) and agarose gel controls. Both microbeads exhibit T2* effects with increasing dosage, with CD3 microbeads showing a weaker effect (FIGS. 1A&1B). The dependency of T2* times in relation to CD3, CD25, and ferumoxytol dosages demonstrates a logarithmic response (FIG. 2). T2* decay is more pronounced in ferumoxytol and CD25 microbead samples compared to CD3 microbeads samples.

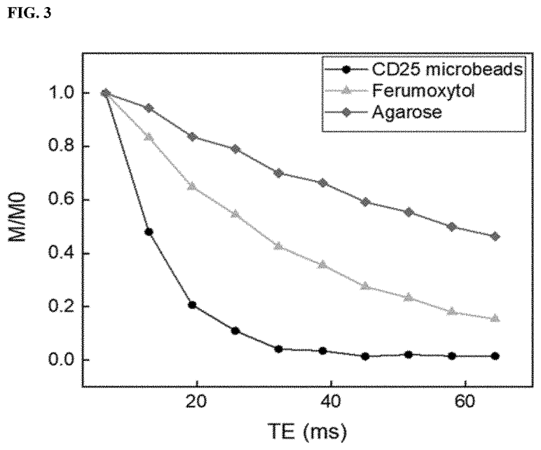

Regarding T2* relaxation curves, both agarose and CD3 microbeads exhibit linear relaxation within the echo time range selected (FIG. 3), indicating long inherent T2* and limited impact of CD3 microbeads on MRI signal. This finding precludes use of CD3 microbeads as a contrast agent. However, ferumoxytol and CD25 microbeads display comparable logarithmic patterns (FIG. 3), demonstrating CD25 microbeads' ability to generate strong signal to noise on MR images.

Cell Internalization of the Microbeads

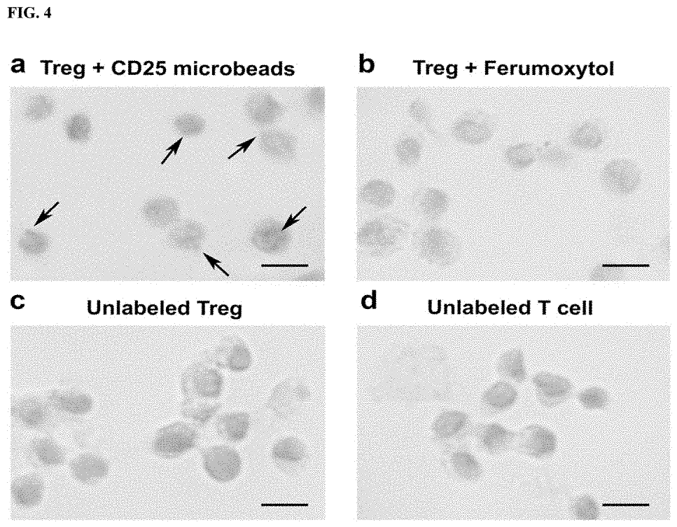

Following MACS sorting, CD25 positive Tregs account for approximately 15% of all CD3 positive isolated T cells. Overnight Treg cell labeling with CD25 microbeads shows positive intracellular Prussian Blue staining, indicating successful uptake of CD25 microbeads (FIG. 4A) without viability impairment (94% viable cells, p<0.01 compared to unlabeled controls). CD25-labeled T cells and unlabeled controls do not exhibit positive staining (FIGS. 4C&4D), signifying that CD25 needs to be present at the surface of the cells to enable internalization of the microbead. Intracellular labeling with CD3 microbeads was unsuccessful, possibly due to the large size of the bead (data not shown) and was therefore excluded from further testing. Likewise, Treg labeling with ferumoxytol is ineffective (FIG. 4B), likely due to the non-phagocytic nature of T cells. These findings reinforce the potential of using CD25 microbeads as a cell tracking agent.

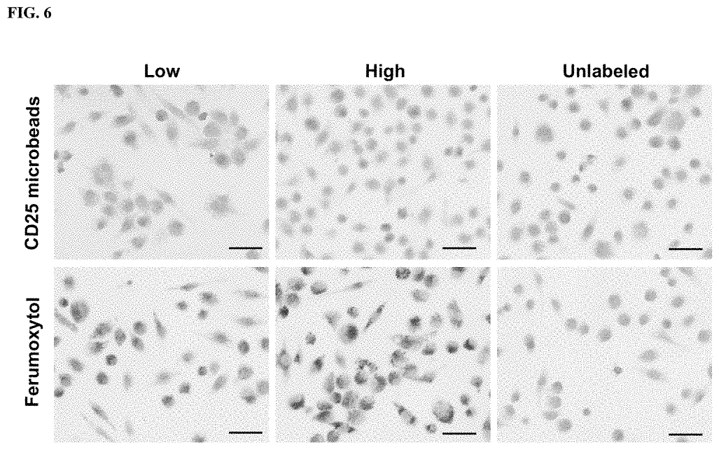

To confirm CD25 microbead specificity towards regulatory T cells, we incubated murine macrophages, an unspecific phagocytic cell type, with either ferumoxytol or CD25 microbeads (FIG. 6). In each case, the findings are reversed compared to Tregs. As expected and previously reported, ferumoxytol is abundantly phagocytosed by macrophages (FIG. 6) as evidenced by the ample intracellular Prussian Blue deposits. CD25 microbeads-labeled macrophages, on the other end, exhibit minimal positive staining (FIG. 6).

MRI of CD25 Microbead-Labeled Tregs

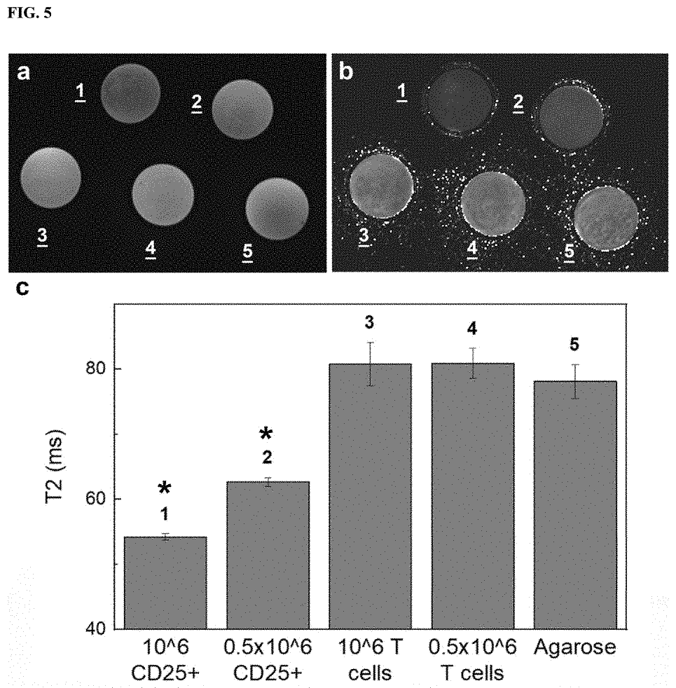

CD25 microbead-labeled Tregs (FIG. 5A, 1-2) displayed decreased signal on T2-weighted MR images compared to unlabeled cells (FIG. 5A, 3-4) and agarose controls (FIG. 5A, 5), consistent with adequate labeling with a paramagnetic iron oxide. T2 relaxation time maps (FIG. 5B) yielded quantitative T2 signal decay. The T2 of half million Tregs (T2₍₂₎=62.67 ms±0.67) is significantly lower and about three-fourth as high as the T2 of unlabeled T cells (T2₍₄₎=80.89 ms±2.34) and agarose alone (T2₍₅₎=78.11 ms±2.59, P=0.0001 and 0.0006 respectively). T2 signal is further shortened in one million labeled Treg samples (T2₍₁₎=54.22 ms±0.51) and significantly lower than the half million labeled Treg samples (P=0.0001).

In Vivo MRI of CD25 Microbead-Labeled Treg.

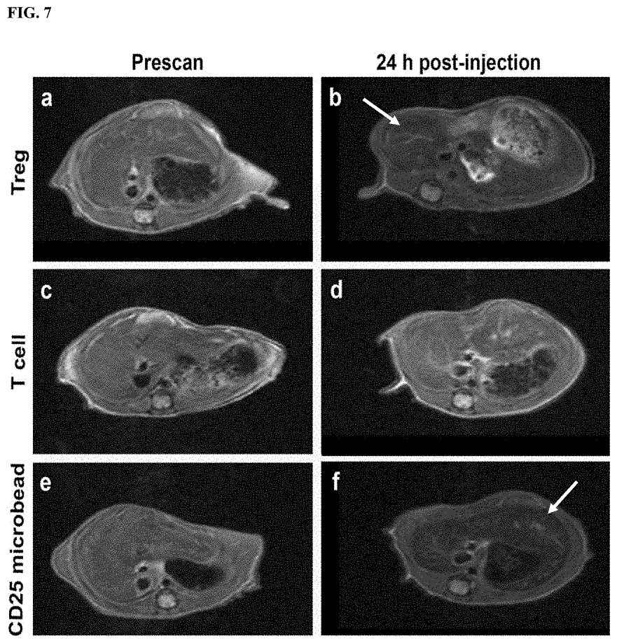

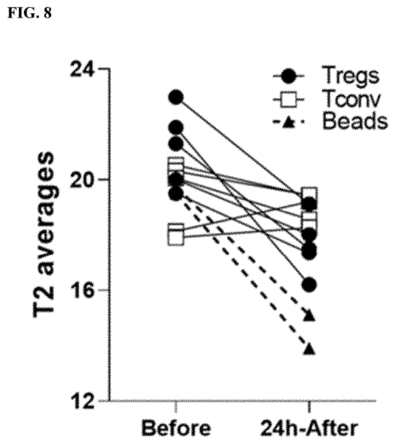

Mice receiving CD25 microbead-labeled Tregs exhibited decreased liver signal on T2-weighted MR images 24 h after systemic injection (FIGS. 7a,b) compared to nega-tive control mice receiving CD25 microbead-labeled T cells (FIGS. 7c,d). Mice receiving CD25 microbeads alone systemically also displayed strong T2 signal decay at 24 h post-injection (FIGS. 7e,f). Quantitative analysis of T2 values in mice receiving CD25 microbead-labeled Tregs showed significant T2 change between pre and 24 h post MRI (p=0.007, FIG. 8). This was not the case in CD25 microbead-labeled T cells (p=0.44, FIG. 8). ANOVA comparing T2 change in all mice confirmed significant signal change in mice receiving Treg compared to T cells and microbeads (p=0.029 and p=0.007 respectively, FIG. 8). These results confirm the feasibility of Treg tracking in vivo.

Discussion

This study demonstrates that the CD25 microbeads used for magnetic sorting of regulatory T cells constitute an excellent cell labeling agent for subsequent MR imaging. The specific antibody binding promotes internalization of the magnetic bead, leading to strong MR effects, which have so far been unattainable with other SPIOs. The successfully labeled T cells with clinically applicable agents like the gold standard Ferumoxytol or the CD25 microbeads described here have not reported previously.

The CD25 microbeads that serve as MRI contrast agents in this project are currently approved for clinical research. CD25 microbeads alone displayed strong MR T2 effects, comparable to the standard Ferumoxytol, which is also used ‘off label’ for cell tracking applications. Equivalent CD34 microbeads are FDA approved as a humanitarian use device, which should fast-track approval for clinical ‘off-label’ use of CD25 microbeads, thereby enabling researchers and clinicians to track regulatory T cells in vivo.

Due to their low cytoplasmic capacity and phagocytic activity, T cells have consistently been challenging to label for MR tracking applications. Yeh et al. (Magn Reson Med 30, 617-625, (1993)) were the first to label T cells with iron oxide particles but the contrast agents used have since been discontinued. Jin et al. (J Cereb Blood Flow Metab 36, 1464-1476, (2016)) reported successful labeling of T cells using Molday Ion, a preclinical ultrasmall SPIO, in a murine stroke model. Garden et al. (J Immunol Methods 314, 123-133, (2006)) ornated their ultrasmall SPIO with TAT peptides to facilitate labeling, yielding both intracytoplasmic and intranuclear labeling. Adding contrast agents and transfection reagents to improve cell labeling presents a major limitation for subsequent FDA approval as adding external agents increases the risk of introducing pathogens and cellular atypia.

When used for labeling Treg cells, CD25 microbeads were able to initiate internalization, likely through the specific CD25 receptor. A significant MR signal change was able to be detected with as few as 5×10⁵ cells in a large voxel. Conversely, Ferumoxytol was not internalized in Tregs. Using the microbeads which are already used for Treg cell sorting can therefore obviate unnecessary cell manipulation with acceptable labeling efficiency. The labeling methods herein would provide one-stop approach where the Treg sorting step also labels the cells which can be readily tracked once they home to specific organs using non-invasive MRI.

While CD25 microbeads specifically labeled Tregs, they were not as readily internalized by macrophages. Macrophages and other phagocytes such as dendritic cells have been an MR cell labeling reference for decades for both practical and clinical reasons. Due to their phagocytic nature, uptake of large amounts of contrast agents is comparatively straightforward thereby enabling elegant non-invasive investigation of immune processes in vivo in cancer, auto-immune and transplant applications. Although peculiar, the finding that CD25 microbeads are not as easily internalized by macrophages offers a desirable edge over the gold standard Ferumoxytol (FIG. 6) for in vivo applications. Uptake of free CD25 contrast from dead Tregs by macrophages resulting in confounding MR signal may therefore be minimized in vivo.

T2*/T2 effects of intracellular iron oxide-based contrast agents (ferumoxytol & CD25 microbeads) can be measured in the tissues where therapeutic cells distribute via creation of T2*/T2 maps. Imaging-based cellular tracking is reliable, reproducible, and non-invasive with MRI. Multiple animal and clinical MRI scanners have in-built multi-echo sequences with T2*/T2 map calculation capability to quantify dynamic change of hypointense clusters in the organ of the interest. One limitation of iron oxide contrast agents is that it only enables semi-quantitative assessment of cell infiltration in a given tissue rather than an absolute number. Fluorinated contrasts agents have shown promise for the labeling and fully quantitative tracking of T cells. Nonetheless, this method requires fluorine-specific MRI hardware and software that is only sparsely available in the clinical setting to date.

Transplant graft rejection is driven by a decline in the frequency of Treg cells or impairment in Treg cell function, which controls alloantigen specific T cells. To prevent graft rejection, shifting the immune balance toward the regulatory arm with Treg cellular therapy has become a very promising therapeutic option. Oo et al. (JHEP Rep 1, 286-296 (2019)) have demonstrated that approximately 22-44% of infused Tregs homed to and were retained in the livers of patients with autoimmune hepatitis for up to 72 h after leukapheresis, labeling, and re-infusion. The authors used indium isotope for labeling and tracked the cells with single photon emission computed tomography (SPECT-CT), which utilizes ionizing radiation. Till date, there are no MRI based cellular tracking methods for Tregs. More importantly, success of Treg homing to the transplant may be an early marker of transplant acceptance and should be determined non-invasively. The current platform which integrates the CD25 sorting agent as a labelling contrast agent may become a key resource in determining homing and fate of Tregs in inflammatory pathologies and solid organ transplantation.

In conclusion, the present CD25 microbead-based labeling method is an effective tool for Treg tagging, yielding detectable MR signal change. Given use of this agent in the existing clinical Treg sorting workflow, this method offers ease of use for Treg labeling during transplant surgery given time and sterility constraints.

CD4 Microbeads In Vitro

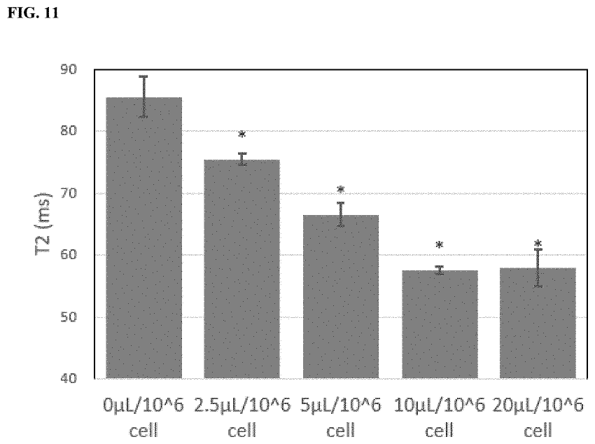

T cells were separated from patient leukapheresis by Ficoll density gradient centrifugation, then negative magnetic cell sorting and positive cell sorting with Cd4 and allowed to expand in vitro for a period of 1 to 14 days. T cells were then labelled with CD4 microbeads at 2×10⁶/mL CD4+ cells and incubated overnight. FIG. 11 depicts quantitative analysis of the T2 shortening in CD4-microbead labeled cells. Data are presented as mean±standard deviation. * indicates p<0.001 compared to agarose control.

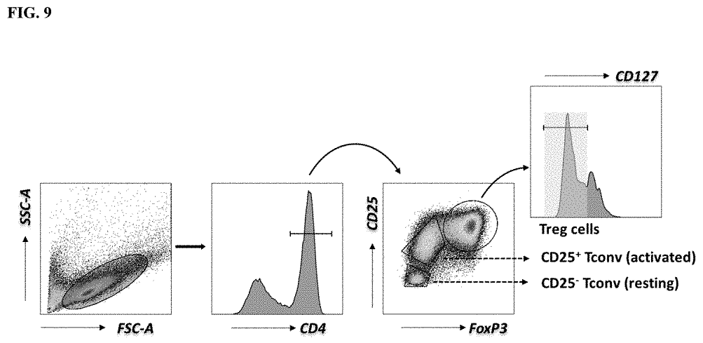

FIG. 9 depicts the sequential gating strategy used to identify resting (CD25⁻) conventional primary human CD4⁺ T cells (Tconv), activated (CD25⁺/FoxP3^{-/LOW}) Tconv and Treg cells from peripheral blood mononuclear cells is based on the differential expression of CD25 and FoxP3 markers in CD4⁺ T cells. The identification of Treg cells required a further analysis of CD127 expression and were identified as CD4⁺/CD25⁺/FoxP3⁺/CD127^{-/LOW}

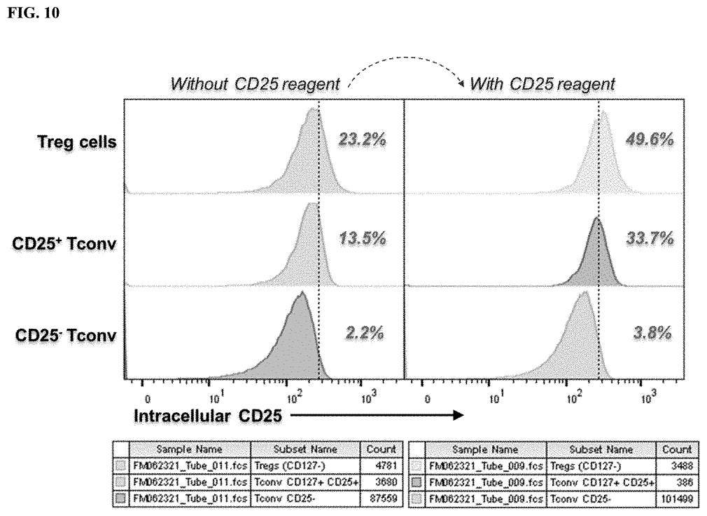

FIG. 10 depicts cargo-induced endocytosis of IL2-R in CD25 nanoparticle-labelled Treg cells. Drug treatments do not affect Treg cell viability. Freshly isolated, non-activated, CD4⁺ T cells were segregated into membrane-expressing CD25⁻ conventional (Tconv), CD25⁺ Tconv and (FoxP3⁺/mCD25⁺/CD127⁻) Treg cells as in FIG. 1. Cells were incubated with or without 1 μL of CD25 reagent (Miltenyi)/10⁶ cells for 10 minutes with slow shaking at 4° C. (same conditions used for Treg cell isolation). After washing off the excess of reagent, cells were left in culture for additional 6 hours, harvested and analyzed by flow cytometry. The results showed that Treg cells that were in the presence of CD25 reagent, expressed twice as much internalized CD25 marker (IL-2 Receptor α subunit) compared to cells without reagent. The same effect was observed in CD25⁺ Tconv cells. The table included illustrates that the data of the different cell types were acquired from the same tube.

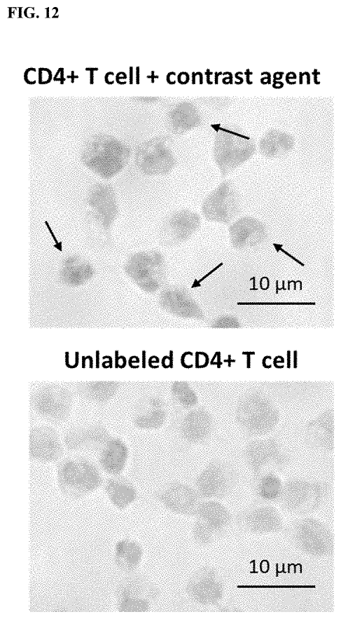

FIG. 12 depicts confirmation of CD4 microbead intracellular uptake by Tregs. Unlabeled control Treg (bottom image) do not exhibit staining.

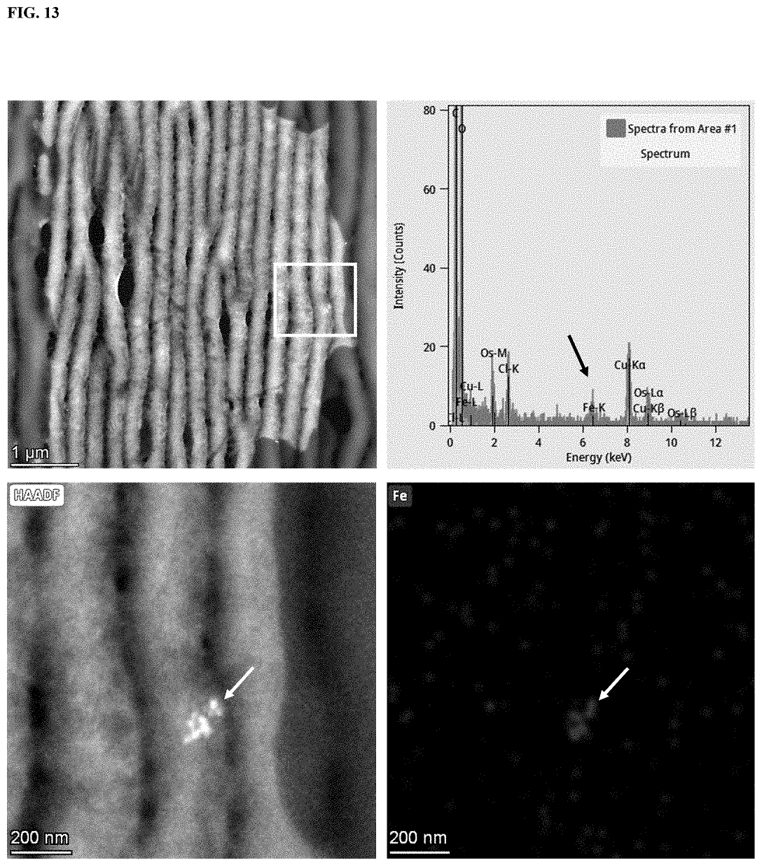

FIG. 13 depicts electron microscopy data confirming intracellular localization of CD4 microbeads. In the top left is presented a full cell image. The bottom left shows the magnified inset thereof. The bottom right shows an iron-specific filter. The top right shows an inset EDS spectrum showing presence of iron in the cell.

Various modifications of the present disclosure, in addition to those shown and described herein, will be apparent to those skilled in the art of the above description. Such modifications are also intended to fall within the scope of the appended claims.

It is appreciated that all reagents are obtainable by sources known in the art unless otherwise specified.

It is also to be understood that this disclosure is not limited to the specific aspects and methods described herein, as specific components and/or conditions may, of course, vary. Furthermore, the terminology used herein is used only for the purpose of describing particular aspects of the present disclosure and is not intended to be limiting in any way. It will be also understood that, although the terms “first,” “second,” “third” etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, “a first element,” “component,” “region,” “layer,” or “section” discussed below could be termed a second (or other) element, component, region, layer, or section without departing from the teachings herein. Similarly, as used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

Unless otherwise defined, all terms (including technical and scientific terms) used herein have the same meaning as commonly understood by one of ordinary skill in the art to which this disclosure belongs. It will be further understood that terms such as those defined in commonly used dictionaries, should be interpreted as having a meaning that is consistent with their meaning in the context of the relevant art and the present disclosure, and will not be interpreted in an idealized or overly formal sense unless expressly so defined herein.

Reference is made in detail to exemplary compositions, aspects and methods of the present disclosure, which constitute the best modes of practicing the disclosure presently known to the inventors. The Figures are not necessarily to scale. However, it is to be understood that the disclosed aspects are merely exemplary of the disclosure that may be embodied in various and alternative forms. Therefore, specific details disclosed herein are not to be interpreted as limiting, but merely as a representative basis for any aspect of the disclosure and/or as a representative basis for teaching one skilled in the art to variously employ the present disclosure.

Patents, publications, and applications mentioned in the specification are indicative of the levels of those skilled in the art to which the disclosure pertains. These patents, publications, and applications are incorporated herein by reference to the same extent as if each individual patent, publication, or application was specifically and individually incorporated herein by reference.

The foregoing description is illustrative of particular embodiments of the disclosure, but is not meant to be a limitation upon the practice thereof. The following claims, including all equivalents thereof, are intended to define the scope of the disclosure.

We claim:

1. A method for obtaining a magnetic resonance image (MRI) of a cell, comprising:
obtaining a cellular sample and binding to one or more cells therein a nanoparticle comprised of an antibody or active fragment thereof to a cell surface protein covalently linked to at least one superparamagnetic iron oxide nanoparticle;
incubating the one or more cells for a period of time from about 5 minutes to 24 hours or more and/or stimulating the one or more cells with an agonist to cause the nanoparticle to be internalized; and,
obtaining at least one MRI of the one or more cells.

2. The method of claim 1, wherein the cellular sample is obtained from a subject.

3. The method of claim 2, further comprising administering the one or more cells back to the subject prior to obtaining the at least one MRI.

4. The method of claim 1, further comprising administering the one or more cells to a subject prior to obtaining the at least one MRI.

5. The method of claim 1, further comprising isolating the one or more cells from the cellular sample.

6. The method of claim 1, wherein the antibody or active fragment thereof binds to CD25.

7. The method of claim 6, wherein the antibody or active fragment thereof binds to human CD25.

8. The method of claim 6, wherein the agonist is CD3, CD28, IL-2, or a combination thereof.

9. The method of claim 1, wherein the antibody or active fragment thereof binds to CD4.

10. The method of claim 9, wherein the antibody or active fragment thereof binds to human CD4.

11. The method of claim 9, wherein the agonist is CD3, CD28, IL-2, or a combination thereof.

12. The method of claim 1, wherein incubating comprises providing a cellular medium and about 37° C. temperature to the one or more cells.

13. The method of claim 1, wherein the one or more cells are T cells.

14. The method of claim 13, wherein the T cells are Treg cells.

15. The method of claim 1, wherein the agonist is covalently linked to a solid support.

16. The method of claim 15, wherein the solid support is a microparticle.

17. A nanoparticle for MRI comprised of a superparamagnetic iron oxide nanoparticle covalently linked to an antibody or active fragment thereof, wherein the antibody or active fragment thereof recognizes a cell surface protein.
